(19) United States
(12) Plant Patent Application Publication
(10) Pub. No.: US 2002/0100099 P1
(43) Pub. Date: Jul. 25, 2002

(54) Guinea impatiens plant named ' Vicky '

(76) Inventor: Norbert Bull, Goennebek (DE)

Correspondence Address: Catherine Anne Whealy, 1513 Melody Lane, Roanoke, TX, 76262, US

(21) Appl. No.: 09/765,321

(22) Filed: Jan. 22, 2001

(51) Int. Cl. A01H 5/00

(52) U.S. Cl. PLT/318

(57) ABSTRACT

A new and distinct cultivar of New Guinea Impatiens plant named ‘Vicky’, characterized by its light pink and bright red bi-colored flowers; moderately large round flowers; freely flowering habit; early to medium flowering response; compact, low-growing and uniformly mounded plant growth habit; very dark green-colored leaves; and resistance to Powdery Mildew.

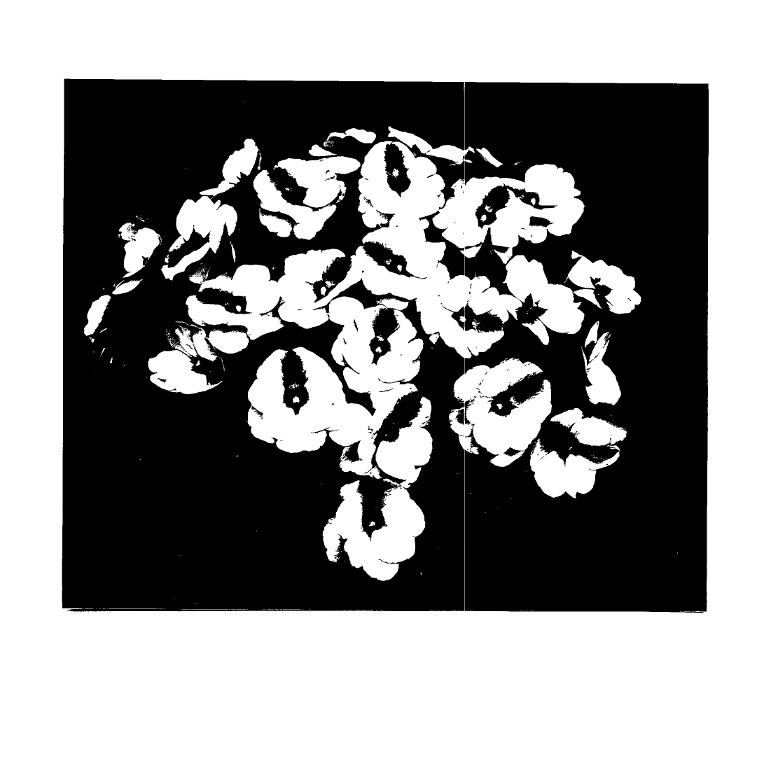

### BACKGROUND OF THE INVENTION

The present Invention relates to a new and distinct cultivar of New Guinea Impatiens plant, botanically known as Impatiens hawkeri, and hereinafter referred to by the name ‘Vicky’.

The new Impatiens is a product of a planned breeding program conducted by the Inventor in Goennebek, Germany. The objective of the breeding program is to develop new Impatiens cultivars with large rounded bi-colored flowers and dark-colored foliage.

The new Impatiens originated from a cross made by the Inventor in 1997 of an Impatiens hawkeri seedling selection designated as code number 2, not patented, as the male, or pollen parent, with an Impatiens hawkeri seedling selection designated as code number 18, not patented, as the female, or seed parent. Both selections are proprietary and used only in the Inventor's breeding program. The cultivar Vicky was discovered and selected by the Inventor as a flowering plant within the progeny of the stated cross in a controlled environment in Goennebek, Germany in the fall of 1997. Plants of the new Impatiens differ from plants of the parent selections primarily in flower and foliage color.

Asexual reproduction of the new cultivar by terminal cuttings taken in Goennebek, Germany, by, or under the supervision of the Inventor, since the fall of 1997, has shown that the unique features of this new Impatiens are stable and reproduced true to type in successive generations.

### SUMMARY OF THE INVENTION

The following traits have been repeatedly observed and are determined to be the unique characteristics of ‘Vicky’. These characteristics in combination distinguish ‘Vicky’ as a new and distinct Impatiens cultivar:

1. Light pink and bright red bi-colored flowers.

2. Moderately large round flowers.

3. Freely flowering habit.

4. Early to medium flowering response.

5. Compact, low-growing and uniformly mounded plant growth habit.

6. Very dark green-colored leaves.

7. Resistant to Powdery Mildew.

Plants of the new Impatiens can be compared to plants of the cultivar Tagula, disclosed in U.S. Plant Pat. No. 10,302. However, in side-by-side comparisons conducted by the Inventor in Goennebek, Germany, plants of the new Impatiens differ from plants of the cultivar Tagula in the following characteristics:

1. Plants of the new Impatiens have smaller leaves than plants of the cultivar Tagula.

2. Flowers of plants of the new Impatiens are positioned above the foliage whereas flowers of plants of the cultivar Tagula are positioned within the foliage.

3. Petals of plants of the new Impatiens are more rounded in shape and apices are not as deeply incised as petals of plants of the cultivar Tagula.

4. Flower color of plants of new Impatiens is light pink and bright red whereas flowers of plants of the cultivar Tagula is close to white and bright red.

Plants of the new Impatiens can also be compared to plants of the cultivar Dark Twilight, not patented. However, in side-by-side comparisons conducted by the Inventor in Goennebek, Germany, plants of the new Impatiens differ from plants of the cultivar Dark Twilight in the following characteristics:

1. Plants of the new Impatiens are more compact and more freely branching than plants of the cultivar Dark Twilight.

2. Leaves of plants of the new Inpatiens are darker green in color than leaves of plants of the cultivar Dark Twilight.

3. Flowers of plants of the new Impatiens have a stronger bicolor contrast but a less distinct star pattern than flowers of plants of the cultivar Dark Twilight.

### BRIEF DESCRIPTION OF THE PHOTOGRAPH

The accompanying colored photograph illustrates the overall appearance of the new cultivar, showing the colors as true as it is reasonably possible to obtain in colored reproductions of this type. Colors in the photograph may differ slightly from the color values cited in the detailed botanical description which accurately describe the colors of the new Impatiens. The photograph comprises a top perspective view of a typical flowering plant of ‘Vicky’ grown in a container.

### DETAILED BOTANICAL DESCRIPTION

The cultivar Vicky has not been observed under all possible environmental conditions. The phenotype may vary somewhat with variations in environment such as temperature, light intensity, daylength, water status and/or fertility level, without, however, any variance in genotype.

The following observations and measurements describe plants grown in Hillscheid, Germany, under commercial practice in a glass-covered greenhouse. Two groups of plants were used for the following description: one group was grown during the spring of 1999 and the other group was grown during the spring of 2000. Both groups of plants were planted in early March in 12-cm containers and grown with 20° C. minimum day/night temperatures. The following observations and measurements were taken when the plants started flowering or about 10 weeks after planting rooted cuttings. In the following description, color references are made to The Royal Horticultural Society Colour Chart except where general terms of ordinary dictionary significance are used.

Botanical Classification: Impatiens hawkeri cultivar Vicky.

Commercial Classification: New Guinea Impatiens cultivar Vicky.

Parentage:

Male parent.—Inventor's proprietary Impatiens hawkeri seedling selection designated as code number 2, not patented.

Female parent.—Inventor's proprietary Impatiens hawkeri seedling selection designated as code number 18, not patented.

Propagation:

Type cutting.—Terminal tip cuttings.

Time to produce rooted cuttings.—About 18 days at 22° C.

Root description.—Numerous, fibrous, and freely branching.

Plant Description:

General appearance.—Compact, low-growing and uniformly mounded plant growth habit; freely branching.

Crop time.—From a rooted cutting, about 10 weeks are required to produce finished flowering plants in 12-cm containers.

Plant height.—About 14 cm.

Plant diameter or spread.—About 27 cm.

Lateral branches.—Internode length: About 1.5 to 1.8 cm. Color: Dark purple, 187A to 187B.

Foliage description.—Arrangement: Primarily in whorls. Length: About 10 cm. Width: About 4 cm. Shape: Elliptic to narrowly ovate. Apex: Slightly acuminate. Base: Acute. Margin: Serrulate with ciliation. Texture: Slightly rugose. Color: Upper surface: Young leaves, very dark purple, between 187A and 200A; fully expanded leaves, very dark green, darker than 139A, glossy; venation, 60B. Lower surface: Mostly very dark purple, close to 187A, with some greenish areas; venation, close to 187A. Petiole: Length: About 1 to 1.2 cm. Color: Upper surface: 60B. Lower surface: 187A.

Flower Description:

Flower type and flowering habit.—Single rounded light pink and bright red bi-colored flowers. Freely flowering, usually about 8 flowers and flower buds per lateral branch. Flowers positioned above and beyond the foliage and typically face upward or outward. Petals self-cleaning; gynoecium persistent. Flowers not fragrant.

Flower longevity.—Flowers last about 3 weeks on the plant.

Flowering season.—Year-round under greenhouse conditions. In the garden, flowering from spring until fall.

Flower length.—About 5.1 cm.

Flower width.—About 5.2 cm.

Petals.—Quantity: Single, five per flower, imbricate. Length: Banner petal: About 1.9 cm. Lateral and base petals: About 2.1 cm. Width: Banner petal: About 3.4 cm. Lateral and base petals: About 3.2 cm. Shape: Roughly cordate. Apex: Emarginate, slightly lobed and somewhat irregular. Base: Attenuate. Margin: Entire. Aspect: Mostly flat, banner petal slightly concave. Texture: Smooth; velvety. Color: When opening and fully opened, upper surface: Banner petal: Central apical splotch, red, 44A; surrounded by light pink, 56A, to margins; base, 46B. Lateral and base petals: Faint central stripe, red, 44A; surrounded by light pink, 56A, to margins; base, 46B. When opening and fully opened, lower surface: Light red, 43B or lighter than 43B.

Spur.—Quantity per flower: One. Length: About 4.2 cm. Aspect: Curved downward. Color: Dark pink, 52A.

Peduncles.—Length: About 4.8 to 5 cm. Strength: Strong, flexible. Aspect: Mostly upright. Color: Light green, 144B, to light red, close to 47A.

Reproductive organs.—Androecium: Stamen number: Five fused at anthers, hooded; filaments free. Anther shape: Obovate. Anther color: Very light yellow, 19D, with small pink, 58B, spots. Pollen color: Light pale yellow, close to 8D. Gynoecium: Pistil quantity: Five. Stigma color: Very dark pink, close to 57C to 57D. Ovary: Five-celled; 4 mm in length. Ovary color: Close to dark purple to black.

Seed development.—Seed development has not been observed.

Disease Resistance: Plants of the new Impatiens have been observed to be resistant to Powdery Mildew.

It is claimed:

1. A new and distinct cultivar of new guinea impatiens plant named ‘vicky’, as illustrated and described:
